(19) United States
(12) Patent Application Publication
(10) Pub. No.: US 2022/0287931 A1
(43) Pub. Date: Sep. 15, 2022

(54) COSMETIC COMPOSITION COMPRISING BENTONITE AS ACTIVE INGREDIENT

(71) Applicant: Korea Institute Of Geoscience And Mineral Resources, Daejeon (KR)

(72) Inventors: Sung Man SEO, Pohang-si (KR); Il-Mo KANG, Seoul (KR); Ki Min ROH, Daejeon (KR); Jae Hwan KIM, Pohang-si (KR); Jeung-Hee AN, Seoul (KR)

(73) Assignee: Korea Institute Of Geoscience And Mineral Resources, Daejeon (KR)

(21) Appl. No.: 17/632,383

(22) PCT Filed: Aug. 6, 2020
(86) PCT No.: PCT/KR2020/010447

§ 371 Date: Feb. 2, 2022

(30) Foreign Application Priority Data
Aug. 9, 2019 (KR) 10-2019-0097407

(51) Int. Cl. A61K 8/26; A61Q 19/00; A61K 8/02; A61Q 19/02

(52) U.S. Cl. CPC A61K 8/26; A61Q 19/007; A61K 8/0266; A61Q 19/02

(57) ABSTRACT

Provided is a cosmetic composition comprising bentonite as an active ingredient. The cosmetic composition according to the present disclosure reduces the expression of inflammatory mechanism proteins and inflammatory mechanism genes in keratinocytes and hypodermal cells, exhibits an anti-inflammatory function, increases the moisture content of the skin, reduces redness in the skin, and increases the brightness of the skin, thereby being effective in skin whitening and skin moisturizing.

### TECHNICAL FIELD

The present disclosure relates to a cosmetic composition comprising bentonite as an active ingredient, and more particularly, to a cosmetic composition capable of reducing the expression of inflammatory mechanism proteins and inflammatory mechanism genes in keratinocytes and hypodermal cells to exhibit an anti-inflammatory function and skin whitening and skin moisturizing functions.

### BACKGROUND ART

In 1925, Coca first described, as atopy, the tendency of dermatitis, asthma, and hay fever to foods and substances exposed through respiratory tract. Atopic diseases include asthma, allergic rhinitis, allergic conjunctivitis, and the like In addition to atopic dermatitis. The incidence rate of atopic dermatitis is rapidly increasing worldwide due to an increase in environmental pollutants, and the prevalence reaches 20% of the total population. Since atopic dermatitis patients have the reduced quality of life due to restrictions in their daily life and increase the economic burden of treatment, measures to manage atopic therapy are urgently needed.

Atopic dermatitis is a recurrent chronic skin disease with severe itching that begins mainly in infancy or childhood, and is a very common skin disease with a family history. Symptoms begin in infancy and childhood, especially around 2 months of age, and about 50% of the symptoms occur before 2 years of age, and most of the symptoms appear before 5 years of age, whereas an example in which the symptoms appear in adults is very rare. In some patients, symptoms are alleviated or spontaneously heal with growth, and more than half of patients who develop in infancy get better before 2 years of age. The shape and distribution of the skin lesions exhibit characteristic prognosis, and pruritus (itching), dry skin, and characteristic eczema are accompanied. In infancy, eczema starts on the face and on the stretched sides of the limbs, but while growing older, the eczema form characteristically appears on the bent part of the arm and the bent part behind the knee. In adults, lichenification occurs when the skin at the folded part is thickened, and eczema frequently occurs on the face, the chest, the nape, etc. other than the limbs compared to the infancy and childhood.

Such atopic dermatitis is a chronic inflammatory skin disease that mainly involves itching and inflammation, and the cause of atopic dermatitis is not clearly known, but it is known that the atopic dermatitis is caused by a complex action of genetic predisposition, immunological factors, and environmental factors. The immunological characteristics reported so far in the occurrence of atopic dermatitis include an increase in the secretion of IgE antibody and IL-4 cytokine in the blood and a decrease in the secretion of IFN-γ cytokine, and in the early disease, T cells that recognize antigens secrete IL-4, IL-5 and IL-cytokines to regulate naive Ca⁴⁺ T cells to be differentiated into Th 2 cells, and the activation of B cells is promoted to increase IgE secretion. Therefore, the levels of IL-4, IL-5, IL-13 cytokine and IgE in the blood of patients with atopic dermatitis appear abnormally high. In addition, Th 1 type cytokines such as IFN-y and IL-12 are decreased. On the other hand, in the case of chronic atopic dermatitis occurring in the late stage, the expression level of IFN-γ cytokine is increased by secondary microbial infection, which leads to an increase in Th 1 cell immune response. IFN-γ cytokine activates macrophages and NK cells, and the activated macrophages secrete cytokines such as TNF-α, IL-6 and IL-β to promote the apoptosis of keratinocytes and cause chronic inflammation such as infiltration into skin tissue. Recently, damage to a skin barrier as well as an immunological mechanism has emerged as a major factor in deterioration of atopic dermatitis. Even in the epidermis, the outermost stratum corneum is formed from keratinocytes and consists of differentiated keratinocytes and lipid layers surrounding them. This skin barrier is easily damaged due to lifestyle habits such as excessive washing or bathing, environmental factors such as dry air and pollutants, and intrinsic diseases such as a decrease in lipid synthesis ability of keratinocytes to cause atopic dermatitis. Therefore, atopic dermatitis shows symptoms such as lichenification, scale, and dryness. In addition, pruritus occurs throughout the acute and chronic phases, which causes aggravation of secondary skin symptoms such as wounds and secondary infection due to scratching, and it is important in the treatment of atopic dermatitis.

Currently, steroidal drugs that suppress the inflammatory reaction and cytokine production are mainly used as a therapeutic agent for atopic dermatitis, but when administered for a long period of time, the steroids cause various side effects, such as skin atrophy or growth delay possibility, and recently, the use of nonsteroidal drugs has been increased. However, nonsteroidal drugs also have various side effects such as symptoms such as erythema, itchiness, edema, erosion and lichenification, and weakening of immunity to make it difficult to treat fundamental atopic dermatitis. Therefore, there is a need for research to find new substances with high therapeutic effects and fewer side effects from natural products with proven safety.

The inventors of the present disclosure found that bentonite itself as a natural substance reduces the expression of inflammation-inducing factors in keratinocytes and hypodermal cells, and proposed a cosmetic composition for alleviating inflammation including atopic dermatitis. Up to now, no example of using bentonite itself to alleviate or treat atopic dermatitis is not known at all, and there is no report on a therapeutic effect of atopic dermatitis.

### DISCLOSURE

### Technical Problem

An object of the present disclosure is to provide a cosmetic composition for anti-inflammation comprising bentonite as an active ingredient.

More specifically, an object of the present disclosure is to provide a cosmetic composition capable of reducing the expression of inflammatory mechanism proteins such as TNF-α, COX-2, IL-6, IL-8, IL-10 and MCP-1 that cause inflammation in the skin to inhibiting the inflammatory response and having an effect of preventing or alleviating atopic dermatitis.

Another object of the present disclosure is to provide a cosmetic composition for skin moisturizing comprising bentonite as an active ingredient.

Yet another object of the present disclosure is to provide a cosmetic composition for skin whitening and skin tone improvement comprising bentonite as an active ingredient.

### Technical Solution

An aspect of the present disclosure provides a cosmetic composition for anti-inflammation comprising bentonite as an active ingredient.

Another aspect of the present disclosure provides a cosmetic composition for skin moisturizing comprising bentonite as an active ingredient.

Yet another aspect of the present disclosure provides a cosmetic composition for skin whitening and skin tone improvement comprising bentonite as an active ingredient.

The cosmetic composition may be prepared as at least one formulation selected from the group consisting of soap, cleansing foam, cleansing cream, cleansing water, bath product, skin lotion, skin softener, skin toner, lotion, cream, essence, astringent, emulsion, gel, lipstick, spray, shampoo, conditioner, treatment, body cleanser, pack, massage agent, face powder, compact, foundation, two-way cake and makeup base for atopy.

The bentonite may be bentonite in which interlayer ions are substituted with calcium (Ca²) ions or bentonite in which interlayer ions are substituted with magnesium (Mg⁺ ions.

The cosmetic composition may reduce the expression of TNF-α, COX-2, IL-6, IL-8, IL-10, and MCP-1 in keratinocytes.

The cosmetic composition may reduce the expression of TNF-α, COX-2, IL-6, IL-8, IL-10, and MCP-1 in hypodermal cells.

### Advantageous Effects

According to the present disclosure, the cosmetic composition for anti-inflammation comprising bentonite as an active ingredient has an effect of reducing the expression of inflammatory mechanism proteins and inflammatory mechanism genes in keratinocytes and hypodermal cells.

In addition, the cosmetic composition for skin moisturizing comprising bentonite as an active ingredient increases the moisture content of the skin, and the cosmetic composition for skin whitening and skin tone improvement is advantageous of reducing redness in the skin and increasing the brightness of the skin.

### DESCRIPTION OF DRAWINGS

FIG. 1 is a graph of comparing cytotoxicity results by concentration in HaCaT keratinocytes and HS68 hypodermal cells of a bentonite sample according to Example 1.

FIG. 2 is a graph of comparing cytotoxicity results by concentration in HaCaT keratinocytes and HS68 hypodermal cells of a bentonite sample according to Example 2.

FIG. 3 is a graph of comparing cytotoxicity results by concentration in HaCaT keratinocytes and HS68 hypodermal cells of a bentonite sample according to Example 3.

FIG. 4 is a graph of comparing anti-inflammatory effects by concentration of bentonite according to Examples 1 and 4 in HaCaT keratinocytes.

FIG. 5 is a graph of comparing anti-inflammatory effects in HaCaT keratinocytes of bentonite samples according to Examples 1 to 3.

FIG. 6 is a graph of comparing anti-inflammatory effects in HaCaT keratinocytes of bentonite samples according to Examples 4 to 6.

FIG. 7 is a graph of comparing anti-inflammatory effects in HS68 hypodermal cells of bentonite samples according to Examples 1 to 3.

FIG. 8 is a graph of comparing anti-inflammatory effects in HS68 hypodermal cells of bentonite samples according to Examples 4 to 6.

FIG. 9 is a graph of comparing atopic dermatitis inhibitory effects in HaCaT keratinocytes of bentonite samples according to Examples 1 to 3.

FIG. 10 is a graph of comparing atopic dermatitis inhibitory effects in HaCaT keratinocytes of bentonite samples according to Examples 4 to 6.

FIG. 11 is a graph of comparing atopic dermatitis inhibitory effects in HS68 hypodermal cells of bentonite samples according to Examples 1 to 3.

FIG. 12 is a graph of comparing atopic dermatitis inhibitory effects in HS68 hypodermal cells of bentonite samples according to Examples 4 to 6.

FIG. 13 is an experimental result of a skin cleansing effect of bentonite samples according to an exemplary embodiment of the present disclosure.

### BEST MODE FOR THE INVENTION

The present disclosure provides a cosmetic composition for anti-inflammation comprising bentonite as an active ingredient.

In addition, the present disclosure provides a cosmetic composition for skin moisturizing comprising bentonite as an active ingredient.

In addition, the present disclosure provides a cosmetic composition for skin whitening and skin tone improvement comprising bentonite as an active ingredient.

The cosmetic composition may be prepared as at least one formulation selected from the group consisting of soap, cleansing foam, cleansing cream, cleansing water, bath product, skin lotion, skin softener, skin toner, lotion, cream, essence, astringent, emulsion, gel, lipstick, spray, shampoo, conditioner, treatment, body cleanser, pack, massage agent, face powder, compact, foundation, two-way cake and makeup base for atopy.

The bentonite may be bentonite in which interlayer ions are substituted with calcium (Ca²) ions or bentonite in which interlayer ions are substituted with magnesium (Mg²⁺ ions.

The cosmetic composition may reduce the expression of TNF-α, COX-2, IL-6, IL-8, IL-10, and MCP-1 in keratinocytes.

The cosmetic composition may reduce the expression of TNF-α, COX-2, IL-6, IL-8, IL-10, and MCP-1 in hypodermal cells.

### MODE FOR THE INVENTION

In the following description, only parts required to understand embodiments of the present disclosure will be described, and it should be noted that the description of other parts will be omitted within a range without departing from the gist of the present disclosure.

Terms and words used in the present specification and claims should not be interpreted as being limited to typical or dictionary meanings, but should be interpreted as meanings and concepts which comply with the technical spirit of the present disclosure, based on the principle that the present inventor can appropriately define the concepts of the terms to describe his/her own invention in the best manner. Therefore, the exemplary embodiments described in the present specification and the configurations illustrated in the drawings are merely the most preferred embodiment of the present disclosure and are not intended to represent all of the technical ideas of the present disclosure, and thus, it should be understood that various equivalents and modifications capable of replacing the exemplary embodiments at the time of this application.

Hereinafter, the present disclosure will be described in detail.

The present disclosure provides a cosmetic composition for anti-inflammation comprising bentonite as an active ingredient.

In addition, the present disclosure provides a cosmetic composition for skin moisturizing comprising bentonite as an active ingredient.

In addition, the present disclosure provides a cosmetic composition for skin whitening and skin tone improvement comprising bentonite as an active ingredient.

In the present disclosure, the bentonite is one of smectite-based clay minerals, and includes a montmorillonite mineral unless otherwise specified.

The bentonite is a kind of clay minerals having montmorillonite as a main constituent mineral, and the names of montmorillonite and bentonite are derived from the Montmorillon district, France and the Port Benton district, Wyoming, USA, and montmorillonite is a mineral belonging to a semi-group in mineralogical classification, and a fine aggregate having a crystal structure of Si-tetrahedral layers and Al-octahedral layers of 2:1.

Ca²⁺ and Mg²⁺ ions are the most common types of cations present between the layers of bentonite, and small amounts of Na⁺, H⁺, and K⁺ exist, and a divalent cation has a stronger binding force with a tetrahedral silica layer than a monovalent cation, and therefore, the degree of swelling is exhibited low when being in contact with water.

The cosmetic composition according to the present disclosure may include the bentonite as an active ingredient, thereby inhibiting an inflammatory reaction, and having effects such as skin moisturizing and skin whitening.

The cosmetic composition according to the present disclosure inhibits the expression of inflammation-related factors such as TNF-α, COX-2, IL-6, IL-8, IL-10, and MCP-1 in keratinocytes. Specifically, according to an exemplary embodiment of the present disclosure, it was confirmed that the expression of TNF-α, COX-2, IL-6, IL-8, IL-10, and MCP-1 induced by LPS was inhibited in an experimental group treated with bentonite.

The cosmetic composition according to the present disclosure inhibits the expression of inflammation-related factors such as TNF-α, COX-2, IL-6, IL-8, IL-10, and MCP-1 in keratinocytes. Specifically, according to an exemplary embodiment of the present disclosure, it was confirmed that the expression of TNF-α, COX-2, IL-6, IL-8, IL-10, and MCP-1 induced by LPS was inhibited in an experimental group treated with bentonite.

The cosmetic composition according to the present disclosure has a skin moisturizing effect, and effects of skin whitening and skin tone improvement. Specifically, according to an exemplary embodiment of the present disclosure, it was confirmed that in human skin treated with bentonite, the skin brightness and yellowness were increased, and the redness was decreased. In addition, as a result of measuring the moisture content in the human skin treated with bentonite, it was confirmed that the moisture content was increased.

In an exemplary embodiment of the present disclosure, the bentonite may use bentonite in which interlayer ions are substituted with calcium (Ca²⁺ ions or bentonite in which interlayer ions are substituted with magnesium (Mg²⁺ ions.

Conventionally, cosmetics containing a bentonite extract and essences, creams, face mists and mask packs applying the cosmetics have been released, but studies related to a therapeutic effect of bentonite itself on atopic dermatitis were insufficient. Accordingly, the present inventors found an inflammatory reaction inhibitory effect and an atopic dermatitis treatment effect of bentonite itself, and confirmed that calcium-based bentonite in which interlayer ions are substituted with calcium (Ca²⁺ ions or magnesium-based bentonite in which interlayer ions are substituted with magnesium (Mg²⁺ ions particularly had these effects, and then completed the present disclosure.

In an exemplary embodiment of the present disclosure, it is preferable that the particle size of the bentonite is 0.01 to 200 μm. If the particle size of the bentonite is less than 0.01 μm, there is a difficulty in a manufacturing process, and since the particle size of the bentonite is too fine, there is a problem that an anti-inflammatory effect and skin whitening and skin tone improvement effects are insignificant. In addition, when the particle size of the bentonite is more than 200 μm, the bentonite may cause irritation to the skin to cause trouble, so that the particle size in the range is preferable.

In the exemplary embodiment of the present disclosure, the bentonite may include 50 to 70 wt% of SiO₂, 10 to 25 wt % of Al₂O₃, 1 to 5 wt % of Fe₂O₃, and 3 to 6 wt % of MgO.

In addition, in the exemplary embodiment of the present disclosure, the bentonite may further include at least one selected from the group consisting of 0 to 5 wt % of CaO, 0 to 5 wt % of K₂O, and 0 to 5 wt % of Na₂O.

The term “active ingredient” used herein means an ingredient that may exhibit a desired activity alone or exhibit activity together with a carrier without activity by itself.

The term “atopic dermatitis” used herein refers to a chronic and recurrent inflammatory skin disease and a disease accompanied by itching, dry skin, and characteristic eczema. The atopic dermatitis is used as the meaning of including all diseases classified as atopic dermatitis in the art regardless of a direct or indirect cause of its occurrence. In general, atopic dermatitis is classified into infant type atopic dermatitis, child type atopic dermatitis, adult type atopic dermatitis, and maternal type atopic dermatitis according to a development time or a subject, and in the present disclosure, atopic dermatitis is defined to include all types of atopic dermatitis described above.

In the present disclosure, the presence of “atopic dermatitis” means a condition in which an infected site of the skin is changed by atopic dermatitis, and this condition includes both a condition considered as skin diseases and a condition not considered as skin diseases.

The “prevention” used herein refers to inhibiting or preventing atopic dermatitis symptoms on the skin by applying the composition of the present disclosure to atopic dermatitis so that the atopic dermatitis symptoms are not generated in advance. In addition, the “alleviation” is used as the meaning including alleviation, prevention, or treatment of atopic dermatitis symptoms.

In the exemplary embodiment of the present disclosure, the composition may be prepared as at least one formulation selected from the group consisting of soap, cleansing foam, cleansing cream, cleansing water, bath product, skin lotion, skin softener, skin toner, lotion, cream, essence, astringent, emulsion, gel, lipstick, spray, shampoo, conditioner, treatment, body cleanser, pack, massage agent, face powder, compact, foundation, two-way cake and makeup base for preventing or alleviating atopic dermatitis, but is not limited thereto.

In the exemplary embodiment of the present disclosure, when the formulation is the paste, cream, or gel, as a carrier ingredient, animal oils, vegetable oils, wax, paraffin, starch, tragacanth, cellulose derivatives, polyethylene glycol, silicone, silica, talc, zinc oxide or the like may be used.

In the exemplary embodiment of the present disclosure, when the formulation is the powder or spray, as the carrier ingredient, lactose, talc, silica, aluminum hydroxide, calcium silicate or polyamide powder may be used, and particularly, in the case of the spray, additionally, a propellant such as chlorofluorohydrocarbon, propane/butane or dimethyl ether may be included.

In the exemplary embodiment of the present disclosure, when the formulation is the solution or emulsion, as the carrier ingredient, a solvent, a solubilizing agent or an emulsifying agent may be used. For example, the carrier ingredient may use water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or fatty acid ester of sorbitan.

In the exemplary embodiment of the present disclosure, when the formulation is the suspension, as the carrier ingredient, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used.

In the exemplary embodiment of the present disclosure, when the formulation of the present disclosure is the surfactant-containing cleansing, as the carrier ingredient, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamido betaine, aliphatic alcohol, fatty acid glyceride, fatty acid diethanolamide, vegetable oil, lanolin derivatives, ethoxylated glycerol fatty acid ester, or the like may be used.

The content of bentonite used in the cosmetic composition is preferably included in an amount of 0.01 to 35 wt % based on 100 wt % of the total cosmetic composition.

When the bentonite is included in an amount of less than 0.01 wt % based on 100 wt % of the cosmetic composition, there is a problem that the anti-inflammatory effect and the skin whitening and skin tone improvement effects are insignificant. When the bentonite is included in an amount of more than 35 wt %, there is a problem in that it is difficult to prepare the cosmetic composition due to a high viscosity problem depending on a physicochemical property (swelling) of bentonite, so that it is preferable to include the bentonite within the range.

### EXAMPLES

Hereinafter, the present disclosure will be described in more detail with reference to Examples. However, these Examples are only illustrative the present disclosure, and the scope of the present disclosure is not limited to these Examples.

### Example 1

### Preparation of Bentonite Sample

In order to measure an anti-inflammatory effect and an atopic dermatitis improvement effect of bentonite, dry powder of local bentonite produced in Yangnam-myeon, Gyeongju was obtained and dissolved in 0.5% DMSO and then diluted again in a culture medium or tertiary distilled water and used in an experiment.

As shown in Table 1 below, bentonite, in which interlayer ions were substituted with calcium ions, was separated into particle sizes of 50 μm or less, 10 to 25 μm, and 0.1 to 0.5 μm, as Examples 1 to 3, and bentonite, in which interlayer ions were substituted with magnesium ions, was separated into particle sizes of 50 μm or less, 10 to 25 μm, and 0.1 to 0.5 μm, as Examples 4 to 6, and an experiment was performed.

TABLE 1

| No. | Sample name | Element | Particle size | |
|---|---|---|---|---|
| Example 1 | Bgp40b-p-Ca · S | Calcium-type | To 50 | μm |
| | | bentonite | | |
| Example 2 | Bgp40b-p-Ca | Calcium-type | 10 to 25 | μm |
| | | bentonite | | |
| Example 3 | Bgp40b-p-Ca · NPs | Calcium-type | 0.1 to 0.5 | μm |
| | | bentonite | | |
| Example 4 | Bgp40b-p-Mg · S | Magnesium-type | To 50 | μm |
| | | bentonite | | |
| Example 5 | Bgp40b-p-Mg | Magnesium-type | 10 to 25 | μm |
| | | bentonite | | |
| Example 6 | Bgp40b-p-Mg · NPs | Magnesium-type | 0.1 to 0.5 | μm |
| | | bentonite | | |

### Example 2

### Preparation of Cells

In order to confirm cytotoxicity evaluation, an anti-inflammatory effect, and an atopic dermatitis inhibitory effect of bentonite, a HaCaT cell line, human-derived keratinocytes, and a HS68 cell line, as human foreskin-derived fibroblasts, were prepared.

Human keratinocytes (HaCaT) were adopted and used from the Korean Cell Line Bank (Seoul, Korea), and hypodermal cells (HS68) were adopted and used from ATCC (American Type Culture Collection, deposit No.: 1635).

### Experiment Example 1

### Cytotoxicity Test of Bentonite

In order to test the cytotoxicity of bentonite samples according to Examples 1 to 3, cytotoxicity was measured in human keratinocytes (HaCaT cells) and human hypodermal cells (HS68 cells).

The cytotoxicity test was a test performed to set an effective treatment concentration of a substance to perform a cell test, and a test for determining a concentration for conducting a stimulation inhibitory effect test of bentonite that can be used in a cosmetic composition in an artificial skin model.

HaCaT keratinocytes and HS68 hypodermal cells were dispensed in a 96-well plate at a concentration of 2×10⁴ cells/well, and then cultured for 24 hours at 37° C. in a 5% CO₂ cell culture condition. After the medium was removed and the cells were washed with phosphate buffered saline (PBS), the cells were maintained in a starvation state for 12 hours, a new medium was added, and bentonite samples were treated by concentration and reacted for 48 hours. To measure the viability of cells, the medium was removed and the cells were washed with PBS, treated with 5 mg/ml of an MTT solution by 20 μl per well, reacted for 4 hours, and then dissolved with dimethyl sulfoxide (DMSO) and then the absorbance was measured at 570 nm.

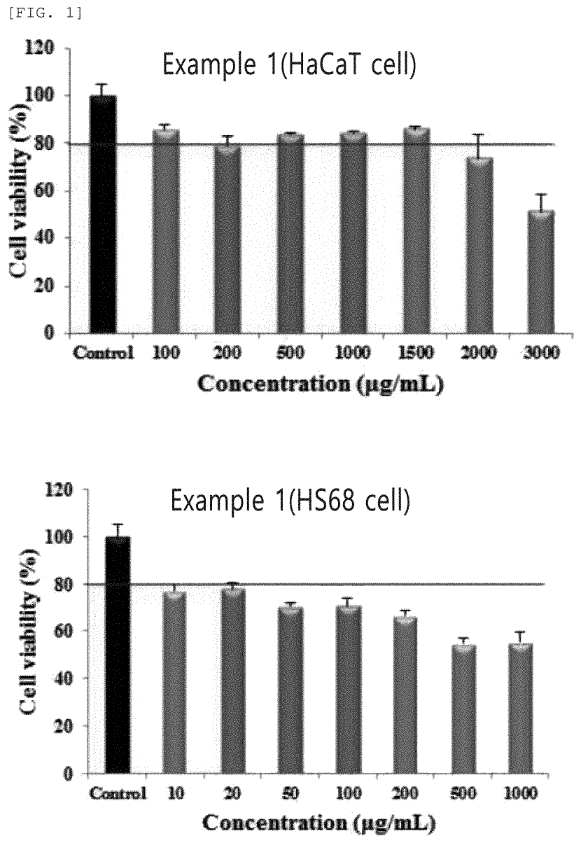

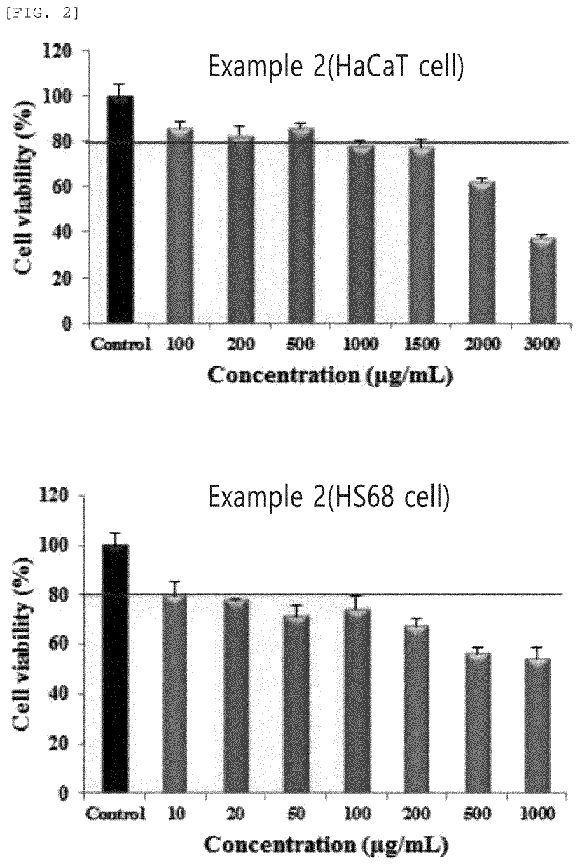

FIG. 1 is a graph of comparing cytotoxicity results by concentration in HaCaT keratinocytes and HS68 hypodermal cells of a bentonite sample according to Example 1, FIG. 2 is a graph of comparing cytotoxicity results by concentration in HaCaT keratinocytes and HS68 hypodermal cells of a bentonite sample according to Example 2, and FIG. is a graph of comparing cytotoxicity results by concentration in HaCaT keratinocytes and HS68 hypodermal cells of a bentonite sample according to Example 3.

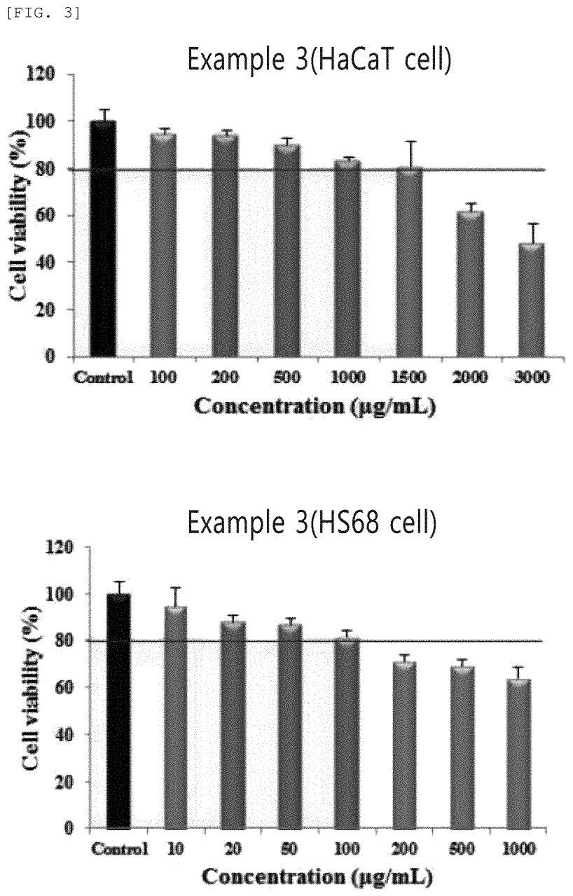

Referring to FIGS. 1 to 3, when bentonite samples of Examples 1 to 3, which were calcium-type bentonite, were at a concentration of 2,000 μg/ml or more in HaCaT keratinocytes, toxicity was recognized as relative viability of 80% or less. In HS68 hypodermal cells, which were relatively sensitive to growth, concentrations with cytotoxity were confirmed as 200, 100, and 100 μg/ml or more, respectively.

Therefore, in a subsequent anti-inflammatory effect confirmation experiment, bentonite treatment was performed at a concentration of 1,000 μg/ml or less in keratinocytes and 100 μg/ml or less in hypodermal cells.

### Experiment Example 2

### Anti-Inflammatory Effect of Bentonite

### Experimental Example 2-1

### Anti-Inflammatory Effect of Bentonite by Concentration

In order to experiment an inflammation improvement effect of bentonite, the bentonite samples of Examples 1 and 4 were mixed with an immunoactive material, lipopolysaccharide (LPS) by concentration, respectively, and reacted in HaCaT keratinocytes for 24 hours, and then the protein expression levels of inflammatory-related genes, TNF-α, COX-2, and IL-6, were tested by western blot (immunoblot).

Before sample treatment, HaCaT keratinocytes were dispensed into an ø90 cell culture dish at a concentration of 1×10⁵ cells/well, stabilized for 24 hours, and then maintained in a starvation state for 12 hours. After pre-treating the bentonite samples of Examples 1 and 4 at concentrations of 200, 100, and 50 μg/ml, an experimental group and a positive control group except for an untreated control group were treated with LPS and reacted for 12 hours. After proteins were isolated from cells and subjected to SDS (Sodium Dodecyl Sulfate)-PAGE (Poly Acrylamide Gel Electrophoresis) electrophoresis, inflammation-related factors TNF-α, COX-2, and IL-6 and a housekeeping gene β-actin were quantitatively analyzed by western blot.

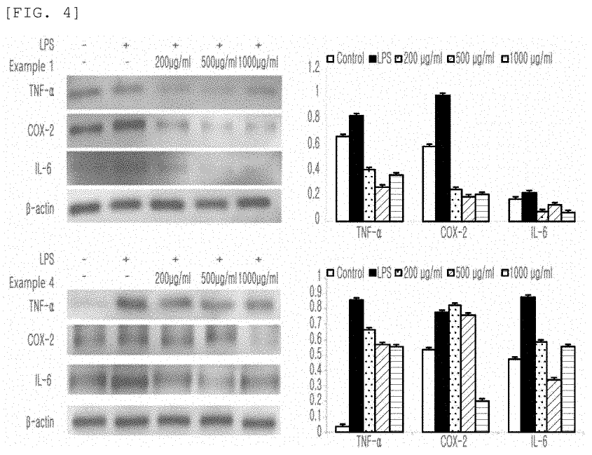

FIG. 4 is a graph of comparing anti-inflammatory effects by concentration of bentonite according to Examples 1 and 4 in HaCaT keratinocytes.

Referring to FIG. 4, as a result of treating the bentonite sample of Example 1, the expression levels of TNF-α, COX-2, IL-6 proteins, which were inflammation-related factors, were reduced in a HaCaT keratinocyte group with inflammation induced by LPS, and as a result of treating the bentonite sample of Example 4, the expression levels of TNF-α, COX-2, IL-6 proteins were reduced in a

HaCaT keratinocyte group with inflammation induced by LPS. Therefore, the anti-inflammatory effects of calcium-type bentonite and magnesium-type bentonite were confirmed in inflammation-induced keratinocytes.

### Experimental Example 2-2

### Anti-Inflammatory Effect by Particle Size of Bentonite in Human-Derived HaCaT Keratinocyte Line

In order to confirm the anti-inflammatory effect by particle size of calcium-type bentonite and magnesium-type bentonite samples in a HaCaT skin keratinocyte line, the bentonite samples of Examples 1 to 6 were treated to immunity-induced HaCaT keratinocytes, and then inflammation-related genes were quantitatively analyzed by western blot technique.

The bentonite samples of Examples 1 to 6 were mixed with immune-inducing substances, cytokines TNF-αand IFN-γ by particle size, respectively, and reacted with HaCaT keratinocytes for 24 hours, and then the expression levels of inflammation-related genes were tested by western blot (immunoblot). Before sample treatment, HaCaT keratinocytes were dispensed into a ø590 cell culture dish at a concentration of 1×10⁵ cells/well, stabilized for 24 hours, and then maintained in a starvation state for 12 hours. After pre-treating the samples of Examples 1 to 6 at a concentration of 100 μg/ml, TNF-α (10 ng/ml) and IFN-γ (10 ng/ml) were treated in the experimental group and the positive control group except for the untreated control group and reacted for 12 hours. After proteins were isolated from cells and subjected to SDS-PAGE electrophoresis, inflammation-related factors TNF-α, COX-2, IL-6, IL-8, IL-10, MCP-1 and a housekeeping gene, β-actin were quantitatively analyzed by western blot.

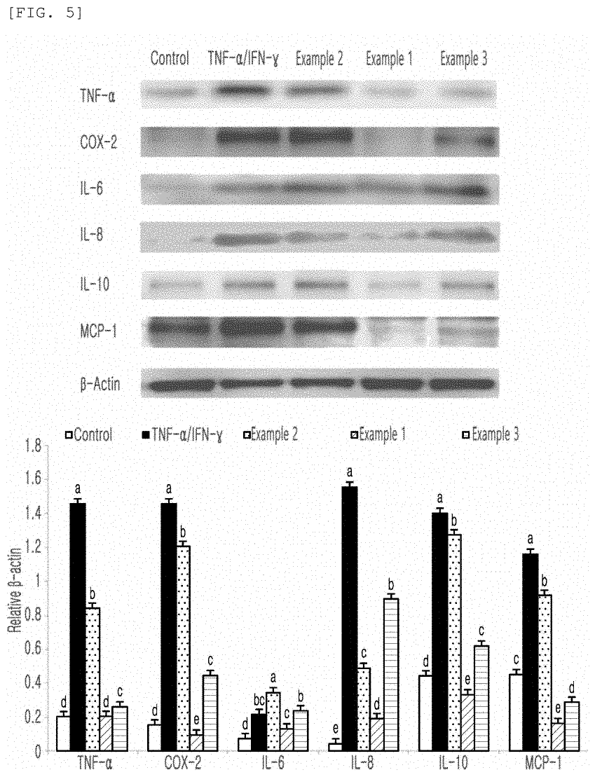

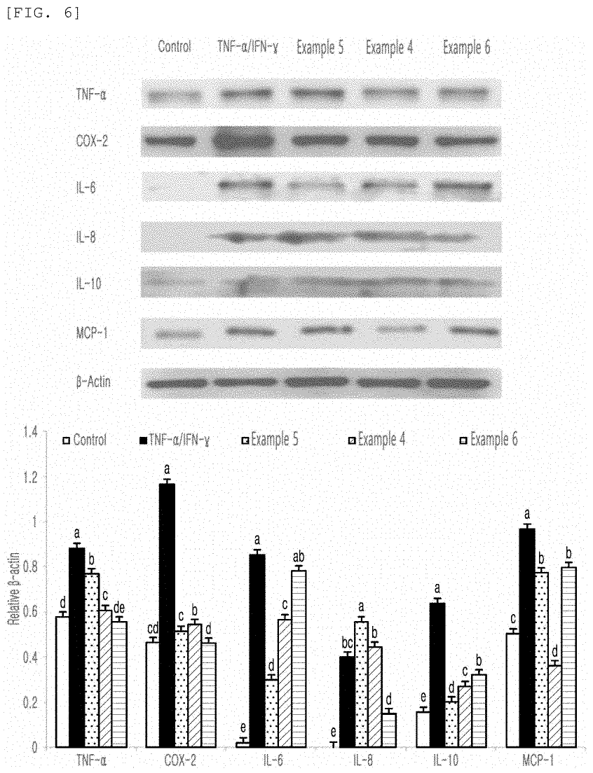

FIG. 5 is a graph of comparing anti-inflammatory effects in HaCaT keratinocytes of bentonite samples according to Examples 1 to 3 and FIG. 6 is a graph of comparing anti-inflammatory effects in HaCaT keratinocytes of bentonite samples according to Examples 4 to 6.

Referring to FIGS. 5 and 6, in the samples having all particle sizes treated with calcium-type bentonite of Examples 1 to 3, the expression of TNF-α, COX-2, IL-6, IL-8, IL-10 and MCP was overall decreased compared to the positive control group. In addition, in the calcium-type bentonite of Example 1 having a particle size of 50 μm or less, the protein expression of immune-related factors was reduced relatively much. In a 0.1 to 0.5 μm of calcium-type bentonite-treated group of Example 3, the expression of all immune factors except for IL-8 was decreased compared to 10 to 25 μm of bentonite of Example 2, but the expression of all immune factors was higher than a case of 50 μm or less.

In the samples of all particle sizes treated with the magnesium-type bentonite of Examples 4 to 6, the expression of inflammation-related factors except for IL-8 was overall reduced compared to the positive control group. Among magnesium bentonite, Example 4 of which the particle size of bentonite was 50 μm or less was confirmed to have the greatest anti-inflammatory effect when showing the decrease width in expression of the MCP-1 immune factor.

### Experimental Example 2-3

### Anti-Inflammatory Effect by Particle Size of Bentonite in Human-Derived HS68 Hypodermal Cell Line

In order to confirm the anti-inflammatory effect by particle size of calcium-type bentonite and magnesium-type bentonite samples in a human-derived HS68 hypodermal cell line, the bentonite samples of Examples 1 to 6 were treated to HS68 hypodermal cells immune-induced by TNF-α/IFN-γ, and then inflammation-related genes were quantitatively analyzed by western blot technique.

The bentonite samples of Examples 1 to 6 were mixed with immune-inducing substances, cytokines TNF-α and IFN-γ by particle size, respectively, and reacted with HS68 hypodermal cells for 24 hours, and then the expression levels of inflammation-related genes were tested by western blot (immunoblot). Before sample treatment, HS68 hypodermal cells were dispensed into a ø90 cell culture dish at a concentration of 1×10⁵ cells/well, stabilized for 24 hours, and then maintained in a starvation state for 12 hours. After pre-treating the samples of Examples 1 to 6 at a concentration of 100 μg/ml, TNF-α (10 ng/ml) and IFN-γ (10 ng/ml) were treated in the experimental group and the positive control group except for the untreated control group and reacted for 12 hours. After proteins were isolated from cells and subjected to SDS-PAGE electrophoresis, inflammation-related factors TNF-α, COX-2, IL-6, IL-8, IL-10, MCP-1 and a housekeeping gene, β-actin were quantitatively analyzed by western blot.

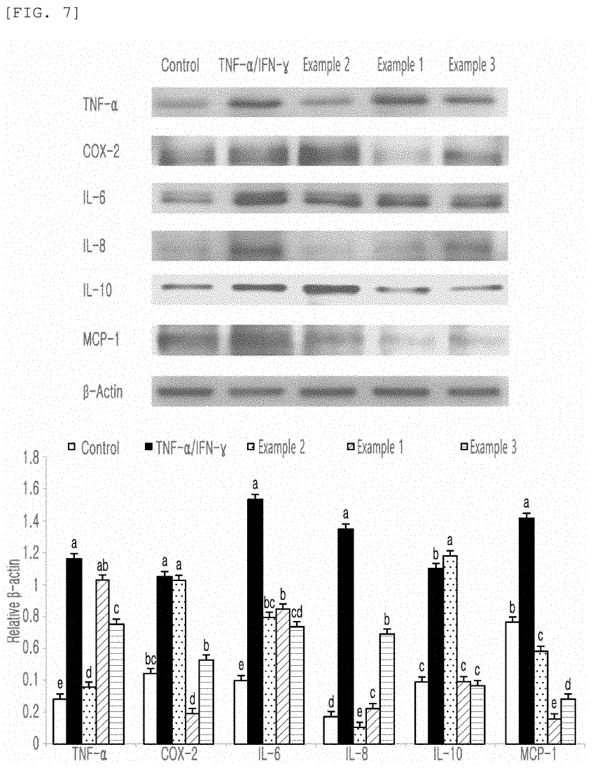

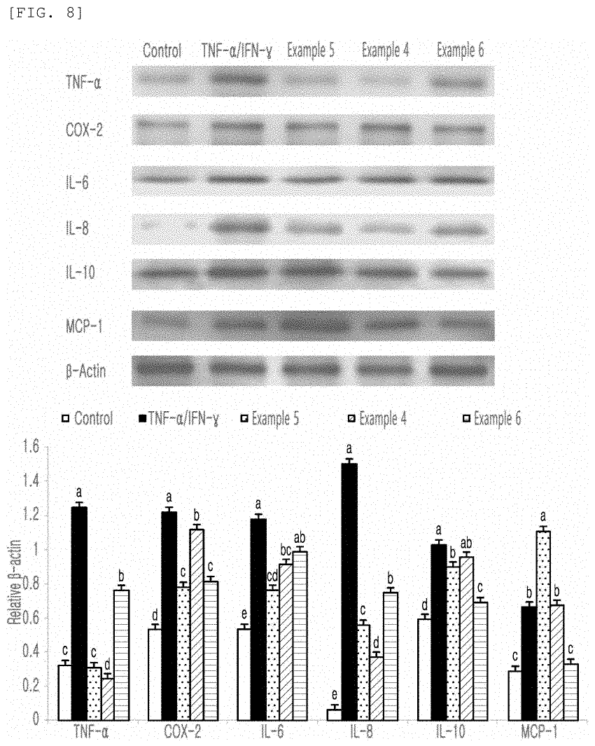

FIG. 7 is a graph of comparing anti-inflammatory effects in HS68 hypodermal cells of bentonite samples according to Examples 1 to 3 and FIG. 8 is a graph of comparing anti-inflammatory effects in HS68 hypodermal cells of bentonite samples according to Examples 4 to 6.

Referring to FIGS. 7 and 8, when the calcium-type bentonite of Examples 1 to 3 was treated, the expression of inflammation-related factors showed a tendency to decrease overall as compared with a positive control group of HS68 hypodermal cells treated only with TNF-α/IFN-γ. In addition, the highest TNF-a expression inhibitory effect was confirmed in the experimental group treated with calcium-type bentonite having a particle size of 10 to 25 μm of Example 2. The IL-10 expression in the same experimental group was significantly increased compared to other experimental groups, and IL-10 was a representative cytokine involved in anti-inflammation and was confirmed by the TNF-α inhibitory effect by the expression of IL-10. In the case of COX-2 and MCP-1, Example 1 showed the lowest expression levels.

When treating the magnesium-type bentonite of Examples 4 to 6, the expression of inflammation-related factors except for MCP-1 was all reduced compared to the positive control group. The TNF-α expression levels of the magnesium-type bentonite of Examples 4 and 5 were reduced to the same level as an untreated negative control group. Considering that calcium-type bentonite with a particle size of 10 to 25 μm exhibited the highest TNF-α expression inhibitory effect, it was confirmed that when the particle size was 10 to 25 μm, TNF-α was the most inhibited in hypodermal cells.

### Experiment Example 3

### Atopic Dermatitis Inhibitory Effect of Bentonite

### Experimental Example 3-1

### Atopic Dermatitis Inhibitory Effect in Human-Derived HaCaT Keratinocyte Line

To confirm the atopic dermatitis inhibitory effect of calcium-type bentonite and magnesium-type bentonite samples in a human-derived HaCaT keratinocyte line, the bentonite of Examples 1 to 6 was treated in cells induced with immune response with TNF-α/IFN-γ and then atopic dermatitis-related genes CCL17 (Chemokine (C-C motif) ligand 17; TARC(thymus-and-activation-regulated chemokine) and CCL21 (Chemokine (C-C motif) ligand 21; MDC(macrophage-derived chemokine)) were quantitatively analyzed by an RT-PCR (Real Time-Polymerase Chain Reaction) technique.

The bentonite samples of Examples 1 to 6 were mixed with immune-inducing substances, cytokines TNF-α and IFN-γ by particle size, respectively, and reacted with HaCaT keratinocytes for 24 hours, and then the expression levels of inflammation-related genes were analyzed by the RT-PCR technique. Before sample treatment, HaCaT keratinocytes were dispensed into a ø90 cell culture dish at a concentration of 1×10⁵ cells/well, stabilized for 24 hours, and then maintained in a starvation state for 12 hours. After pre-treating the bentonite samples at a concentration of 100 μg/ml, TNF-α (10 ng/ml) and IFN-γ (10 ng/ml) were treated in an experimental group and a positive control group except for an untreated control group and reacted for 12 hours. The untreated experimental group was set as a negative control group, and an experimental group treated with TNF-α/IFN-γ alone was set as a positive control group. After 12 hours of reaction, total RNA was isolated from the cells using a Trizol® reagent, and then reverse-transcribed using a random hexamer and Superscript II to synthesize cDNA. The synthesized cDNA was used as a template, and the expression levels of atopic dermatitis-related genes CCL17 and CCL21 and a housekeeping genes GAPDH (Glyceraldehyde-3-phosphate dehydrogenase) were examined by a PCR method.

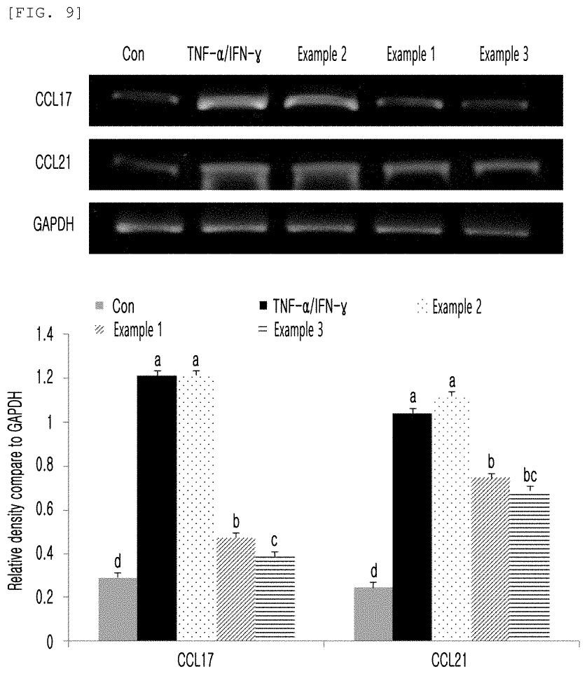

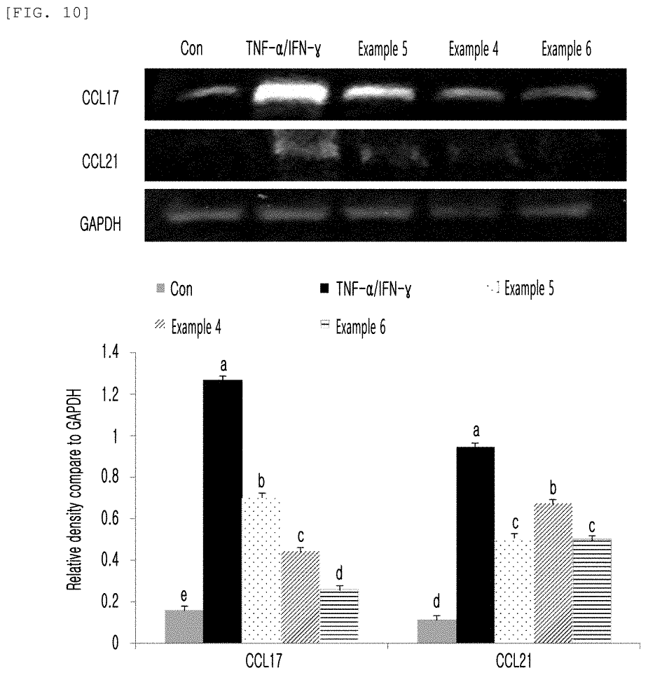

FIG. 9 is a graph of comparing atopic dermatitis inhibitory effects in HaCaT keratinocytes of bentonite samples according to Examples 1 to 3 and FIG. 10 is a graph of comparing atopic dermatitis inhibitory effects in HaCaT keratinocytes of bentonite samples according to Examples 4 to 6.

Referring to FIGS. 9 and 10, in HaCaT keratinocytes treated with the calcium-type bentonite of Examples 1 and 3, the expression level OF CCL17 mRNA was reduced by 2.56 times and 3.12 times compared to the inflammation-induced positive control group, respectively, and the expression of CCL21 mRNA was decreased by 1.39 times and 1.76 times compared to the positive control group, respectively.

In addition, when the magnesium-type bentonite of Examples 4 to 6 was treated, the expression level of CCL17 was decreased 1.80 to 4.92 times, and the expression of CCL21 mRNA was also decreased in all experimental groups. Therefore, it was confirmed that the bentonite of Examples 1 and 3 to 6 inhibited the expression of atopic dermatitis-related factors in human-derived keratinocytes, and among them, it was confirmed that calcium-type bentonite having a particle size of 0.1 to 0.5 μm was the most effective.

### Experimental Example 3-2

### Atopic Dermatitis Inhibitory Effect in Human-Derived HS68 Hypodermal Cell Line

To confirm an atopic dermatitis inhibitory effect of calcium-type bentonite and magnesium-type bentonite samples in a human-derived HS68 hypodermal cell line, the bentonite samples of Examples 1 to 6 were treated in cells induced with immune response with TNF-α/IFN-γ and then atopic dermatitis-related genes CCL17 and CCL21 were quantitatively analyzed by an RT-PCR technique. The experiment was conducted in the same manner as the experimental method using the HaCaT keratinocyte line of Experimental Example 3-1.

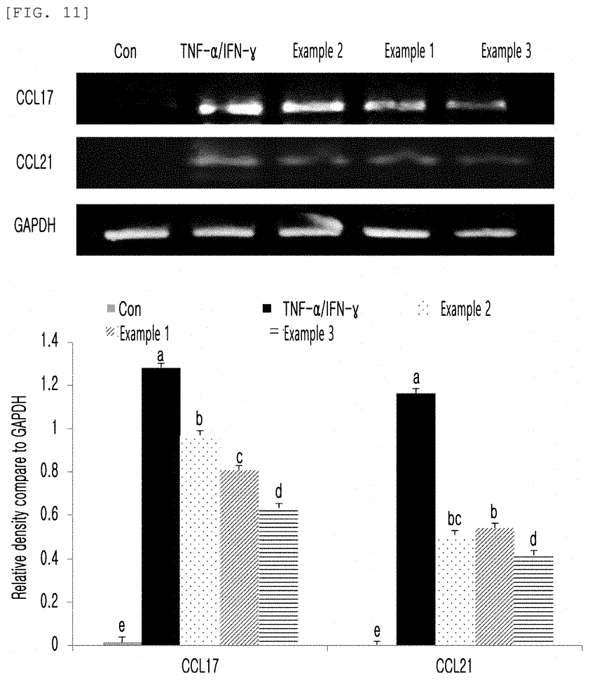

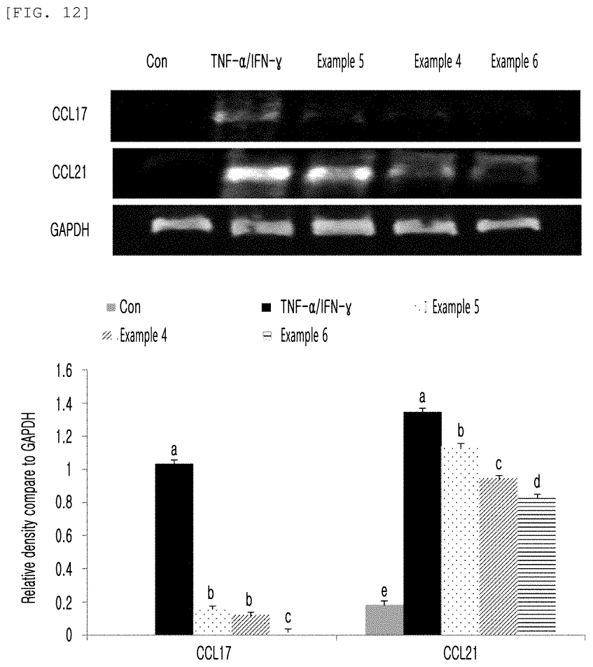

FIG. 11 is a graph of comparing atopic dermatitis inhibitory effects in HS68 hypodermal cells of bentonite samples according to Examples 1 to 3 and FIG. 12 is a graph of comparing atopic dermatitis inhibitory effects in HS68 hypodermal cells of bentonite samples according to Examples 4 to 6.

Referring to FIGS. 11 and 12, it was observed that in HS68 hypodermal cells, compared to a positive control group treated only with TNF-α/IFN-γ, the expression level of CCL17 mRNA was decreased in all experimental groups treated with the calcium-type bentonite of Examples 1 to 3. The expression level of CCL21 mRNA was also decreased by 2.29 times and 2.80 times in Examples 2 and 3, respectively.

Even in the case of magnesium-type bentonite, the expression of CCL17 was inhibited in all experimental groups compared to the positive control group. The expression level of CCL21 mRNA was also decreased compared to the positive control group, and the most inhibitory effect was confirmed when the particle size was 0.1 to 0.5 μm.

### Experiment Example 4

### Skin Cleansing Test of Bentonite

### Experimental Example 4-1

### Cleansing Test of Bentonite-Containing Cleansing Product

A human efficacy test of a bentonite-containing cleansing product was conducted on 10 test participants.

After the cleansing product was applied to a test site, the skin color changes of the test site were evaluated using a chromameter. In addition, visual evaluation of skin photographs was performed using polarized dermoscopy. The test was conducted once a day for 2 weeks by applying the cleansing product to the left arm. As a control group, the non-washed right arm was comparatively evaluated.

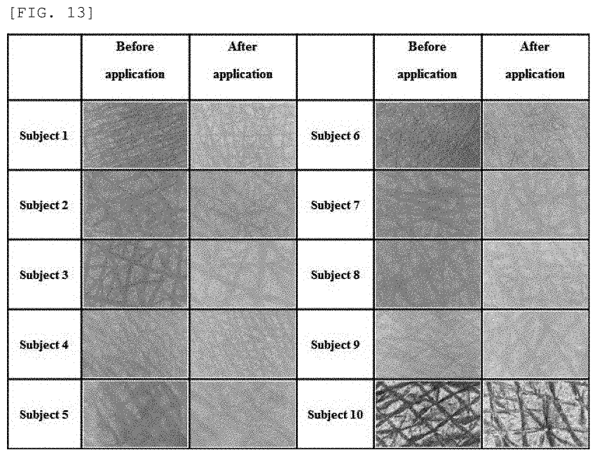

FIG. 13 is an experimental result of a skin cleansing effect of bentonite samples according to an exemplary embodiment of the present disclosure.

Referring to FIG. 13, as a result of measuring the skin brightness and yellowness of the inner arm skin in 10 test subjects, after the test was conducted for 2 weeks, the skin brightness increased by about average 0.77, and no significant difference was observed in the yellowness. As a result of measuring the redness of the skin, about average 0.31 was decreased, and the moisture content of the skin was increased by average 1.523%.

### Experimental Example 4-2

### Cleansing Test of Bentonite Itself

In Experimental Example 4-1, in order to confirm that the whitening and moisturizing effects of the cleansing product was an effect due to bentonite itself contained in the product, the cleaning of only the bentonite raw material was tested. A human efficacy test was performed in the same manner as in Experimental Example 4-1 using bentonite of 5, 10, and 25% (w/v) as a test substance for 10 test participants.

Tables 2 to 4 below were tables summarizing the results of the human efficacy test using 5, 10, and 25% (w/v) of bentonite as the test substance, respectively.

TABLE 2

| | Average before | Average after | Average of |
|---|---|---|---|
| | application | application | variation |
| Lightness | 67.27 | 68.08 | 0.814 |
| Redness | 9.11 | 8.65 | −0.46 |
| Yellowness | 26.29 | 26.97 | 0.679 |
| Moisture content | 11.91 | 12.97 | 1.062 |

TABLE 3

| | Average before | Average after | Average of |
|---|---|---|---|
| | application | application | variation |
| Lightness | 67.87 | 68.02 | 0.156 |
| Redness | 8.96 | 8.70 | −0.265 |
| Yellowness | 27.14 | 27.36 | 0.219 |
| Moisture content | 11.57 | 12.96 | 1.389 |

TABLE 4

| | Average before | Average after | Average of |
|---|---|---|---|
| | application | application | variation |
| Lightness | 67.46 | 68.11 | 0.651 |
| Redness | 8.75 | 8.22 | −0.536 |
| Yellowness | 26.67 | 27.48 | 0.805 |
| Moisture content | 11.84 | 12.59 | 0.746 |

The test was stopped because a red rash developed on the inside of the left arm of one sensitive test subject of the 10 test subjects. Referring to Tables 2 to 4, as a result of measuring the skin brightness and yellowness of the inner arm skin in 9 test subjects, the skin brightness and the yellowness were entirely increased after the test was conducted for 2 weeks. In particular, the skin brightness of 5% (w/v) bentonite was the most increased, and the yellowness of 20% (w/v) bentonite was the most increased.

As a result of measuring the redness, the overall redness was decreased after the test for 2 weeks, and in particular, the redness of 20% (w/v) bentonite was the most reduced. As a result of measuring the moisture content, the overall moisture content was increased after the test for 2 weeks, and in particular, the moisture content of 10% (w/v) bentonite was the most increased.

As a result of the visual evaluation of skin photographs, it was confirmed that the skin brightness of 9 test subjects was increased in the skin using all concentrations of bentonite.

As descried above, specific examples of the cosmetic composition for preventing or alleviating atopic dermatitis comprising bentonite as the active ingredient according to the exemplary embodiment of the present disclosure have been described, but it will be apparent that various modifications are possible without departing from the scope of the invention.

Therefore, the scope of the present disclosure should not be limited to the embodiments and should be defined by the appended claims and equivalents to the appended claims.

In other words, the embodiments described above are illustrative in all aspects and should be understood as not being restrictive, and the scope of the present disclosure is represented by appended claims to be described below rather than the detailed description, and it is to be interpreted that the meaning and scope of the appended claims and all changed or modified forms derived from the equivalents thereof are included within the scope of the present disclosure.

### INDUSTRIAL AVAILABILITY

A cosmetic composition for anti-inflammation comprising bentonite as an active ingredient according to the present disclosure has an effect of reducing the expression of inflammatory mechanism proteins and inflammatory mechanism genes in keratinocytes and hypodermal cells.

In addition, a cosmetic composition for skin moisturizing comprising bentonite as an active ingredient according to the present disclosure increases the moisture content of the skin, and a cosmetic composition for skin whitening and skin tone improvement has an advantageous effect of reducing redness in the skin and increasing the brightness of the skin.

1-7. (canceled)

8. A cosmetic composition for anti-inflammation comprising bentonite as an active ingredient and having an effect on inflammation including atopic dermatitis.

9. A cosmetic composition for skin moisturizing comprising bentonite as an active ingredient.

10. A cosmetic composition for skin whitening and skin tone improvement comprising bentonite as an active ingredient.

11. The cosmetic composition comprising bentonite as the active ingredient of claims 8, wherein the cosmetic composition is prepared as at least one formulation selected from the group consisting of soap, cleansing foam, cleansing cream, cleansing water, bath product, skin lotion, skin softener, skin toner, lotion, cream, essence, astringent, emulsion, gel, lipstick, spray, shampoo, conditioner, treatment, body cleanser, pack, massage agent, face powder, compact, foundation, two-way cake and makeup base for atopy.

12. The cosmetic composition comprising bentonite as the active ingredient of claims 9, wherein the cosmetic composition is prepared as at least one formulation selected from the group consisting of soap, cleansing foam, cleansing cream, cleansing water, bath product, skin lotion, skin softener, skin toner, lotion, cream, essence, astringent, emulsion, gel, lipstick, spray, shampoo, conditioner, treatment, body cleanser, pack, massage agent, face powder, compact, foundation, two-way cake and makeup base for atopy.

13. The cosmetic composition comprising bentonite as the active ingredient of claims 10, wherein the cosmetic composition is prepared as at least one formulation selected from the group consisting of soap, cleansing foam, cleansing cream, cleansing water, bath product, skin lotion, skin softener, skin toner, lotion, cream, essence, astringent, emulsion, gel, lipstick, spray, shampoo, conditioner, treatment, body cleanser, pack, massage agent, face powder, compact, foundation, two-way cake and makeup base for atopy.

14. The cosmetic composition for anti-inflammation comprising bentonite as the active ingredient of claim 8, wherein the bentonite is bentonite in which interlayer ions are substituted with calcium (Ca²⁺) ions or bentonite in which interlayer ions are substituted with magnesium (Mg²⁺) ions.

15. The cosmetic composition for anti-inflammation comprising bentonite as the active ingredient of claim 8, wherein the cosmetic composition reduces the expression of TNF-α, COX-2, IL-6, IL-8, IL-10, and MCP-1 in keratinocytes.

16. The cosmetic composition for anti-inflammation comprising bentonite as the active ingredient of claim 8, wherein the cosmetic composition reduces the expression of TNF-α, COX-2, IL-6, IL-8, IL-10, and MCP-1 in hypodermal cells.
